(19) United States
(12) Patent Application Publication
(10) Pub. No.: US 2002/0142940 A1
(43) Pub. Date: Oct. 3, 2002

(54) Method of inhibiting viral infection using HMG-COA reductase inhibitors and isoprenylation inhibitors

(76) Inventors: Barney Scott Graham, Rockville, MD (US); Tara L. Gower, Nashville, TN (US); Manoj K. Pastey, Silver Spring, MD (US)

Correspondence Address: FULBRIGHT & JAWORSKI L.L.P., A REGISTERED LIMITED PARTNERSHIP, SUITE 2400, 600 CONGRESS AVENUE, AUSTIN, TX, 78701, US

(21) Appl. No.: 09/981,682

(22) Filed: Oct. 16, 2001

Related U.S. Application Data
(60) Provisional application No. 60/241,247, filed on Oct. 17, 2000.

(51) Int. Cl. A61K 31/00

(52) U.S. Cl. 514/1

(57) ABSTRACT

Applicants provide methods of inhibiting viral infections, and treating an infected individual with AIDS, respiratory syncytial virus infection, parainfluenza virus infection, and other viral infections. Inhibitors of Rho isoprenylation are used to inhibit Rho cell surface attachment, thereby inhibiting the use, by viruses, of Rho as a receptor for infection of susceptible cells. Isoprenylation inhibitors include inhibitors specific for the enzymes farnesyltransferase and geranylgeranyltranferase, as well as inhibitors of general cholesterol biosynthesis, such as HMG-CoA reductase inhibitors.

A portion of the work described herein was funded in part under grant no. AID 33933 awarded by The National Institutes of Health. The government has certain rights in the invention.

### BACKGROUND OF THE INVENTION

1. Field of the Invention

The present invention relates to methods of inhibiting viral infection in a mammalian subject. More specifically, the present invention relates to methods of inhibiting viral infections of mammalian cells.

2. Description of Related Art

The Human Immunodeficiency Virus (HIV) epidemic continues to grow at a rapid rate, and the clinical manifestations associated with this viral infection present increasingly more complex medical and socioeconomic problems. Acute HIV infection leads to a period of rapid viral replication, followed by viremia that results in infection of 1% or more of circulating T lymphocytes, the primary target of the virus. Viremia is transient, however, because the cells infected with HIV are removed from circulation by an effective host immune response that results in a 10-to 100-fold decrease in the HIV-infected T cells. Although the initial host response is effective in reducing and controlling HIV-infected cell numbers, it is not sufficient to prevent the postintegration latent or low-level-persistent (LLP) asymptomatic infections of host reservoir cells, such as circulating CD4+T lymphocytes and monocyte/macrophages.

No prevention or cure has yet been found for HIV infection. Current treatments for HIV infection attempt to retard the progress of the disease or relieve its symptoms. Treatment in use today include certain dideoxynucleotides such as azidothymidine (AZT or zidovudine, Burroughs Wellcome), dideoxyinosine (ddI, Bristol-Myers Squibb) or dideoxycytidine (ddC, Hoffinan-LaRoche). However, these agents can be toxic, and their applicability is limited because of the appearance in some patients of onerous, and sometimes lethal, side effects. These side effects include myelosuppression, peripheral neuropathy, and pancreatitis. In some patients, AZT loses effectiveness after prolonged use. While other drugs have been proposed for treatment of HIV infection, including the recent introduction of several HIV protease inhibitors, none have yet been demonstrated to be completely effective. Therefore, there remains a need in the art to develop additional therapeutic agents to prevent and treat HIV infection.

Other viruses can be life threatening for those patients who are already immunocompromised. For example, 40 to 60% of bone marrow transplant patients develop pneumonia often the result of a viral infection. Respiratory syncytial virus (RSV) infection is a common cause of such pneumonias. The problem is significant, as illustrated by the fact that during a 9-week period beginning Jan. 8, 1993, and ending Mar. 4, 1993, 38% of 111 hospitalized adult bone marrow transplant recipients at M.D. Anderson Cancer Center exhibited symptoms of acute respiratory illness. RSV was isolated from half these patients (Whimbey et al., 1995).

Parainfluenza virus, especially parainfluenza virus type III (PIV-3) also is a cause of serious lower respiratory tract disease in both adults and children who undergo bone marrow transplantation (Wendt et al., 1992). Both RSV and PIV constitute a threat to any immunocompromised patient. Current treatment for these viral infections consists primarily of ribivarin, a broad-spectrum antiviral, occasionally augmented with intravenous immunoglobulin therapy.

No vaccine currently exists for HIV, RSV or PIV-3 either, but even if a vaccine were available, these viruses would still constitute a threat to patients in whom the immune system has been either naturally or artificially suppressed. Moreover, no vaccine is 100% effective. For patients suffering from any of these viruses, a more effective therapy is needed.

### SUMMARY OF THE INVENTION

The present invention relates to a method of using inhibitors of HMG-CoA reductase and isoprenylation to inhibit a cellular entry receptor for a number of viruses, including HIV-1, RSV and Parainfluenza virus-3 (PI-3). In one embodiment, inhibitors of HMG-CoA reductase and/or isoprenylation are used prophylactically to prevent infection by viruses that utilize the RhoA receptor. A number of viruses have been demonstrated to have sequence homology in their fusion, envelope, or hemagglutinin proteins, making them likely to utilize the RhoA receptor. Among those sharing sequence homology with RSV, HIV, and PIV-3 are the Ebola virus, Sendai virus, Moloney leukemia virus, and equine infectious anemia virus (Chambers et al., 1990). All viruses in the Paramyxoviridae, Lentivirus and Filovirus families, including both human and veterinary pathogens, share a common structure and function of their fusion glycoprotein which mediates the events inhibited by the HMG-CoA reductase and isoprenylation inhibitors. Each of the viruses encompassed by these familrs is, therefore, included within the scope of the present invention as being suitable targets for both therapy and prevention.

In another embodiment of the invention, one or more inhibitors of HMG-CoA reductase are administered to a subject diagnosed with an existing viral infection to prevent the spread of the infection from cell to cell within the body. Alternatively, one or more inhibitors of isoprenylation, including farnesyl transferase (FT) inhibitors and/or geranylgeranyl transferase inhibitors (GGT) are administered to a patient diagnosed with a viral infection to prevent the spread of the virus within the body. Subjects for whom such treatment is indicated are patients who have not yet begun a regimen of viral treatment, patients who are not currently participating in a viral treatment regimen (often because of the failure of previous treatment), and patients for whom the method of the present invention constitutes a component of a combination therapy for the treatment of viral infection. Other embodiments include those combining HMG-CoA reductase inhibitors and isoprenylation inhibitors. HIV, RSV and PIV are particular targets for these therapies.

In a more particular embodiment of the present invention, either HMG-CoA reductase inhibitors or isoprenylation inhibitors (inhibitors of farnesylation or geranylgeranylation) are administered to a patient who has recently undergone transplant surgery in order to decrease the risk of infection with opportunistic viruses, for example, RSV or Parainfluenza. These patients may include organ transplant recipients and bone marrow transplant recipients, in whom RSV or Parainfluenza infection is common and often fatal. Inhibition of isoprenylation, and concomittant inhibition of cell surface expression of RhoA, inhibits viral infection of susceptible cells and viral spread from infected cells to uninfected cells.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Lovastatin diminishes RSV replication in mice. BALB/c mice were given 1 mg/day lovastatin, 50 mg/day gemfibrozil or PBS by oral gavage starting three days prior to RSV or vaccinia virus infection and for 8 days after infection. Mice were infected intranasally with RSV (hatched bars) or vaccinia virus (solid bars) at day 0. Lungs were harvested at day 4 for RSV and vaccinia plaque assays. Each group represents 5 mice and error bars represent standard deviation.

FIG. 2—Inhibition by lovastatin is dependent on the duration of the treatment. BALB/c mice were given 1 mg/day of lovastatin by oral gavage at several time points throughout the course of RSV infection beginning either: three days prior to RSV infection, one day prior to infection, one day post infection, or three days post infection. Control mice were infected with RSV but not treated with lovastatin. Lungs were harvested 4 days after RSV infection for viral plaque assay. Each group represents 5 mice and error bars represent standard deviation.

FIG. 3—Lovastatin diminishes RSV-induced weight loss in mice. BALB/c mice were given 1 mg/ day of lovastatin beginning either: three days prior to RSV infection, one day prior to infection, one day post infection, or three days post infection. Untreated mice were also infected with RSV. Mice were weighed daily and percent weight loss was calculated. Each group represents 3 mice and error bars represent standard deviation.

FIG. 4—Lovastatin does not effect serum cholesterol levels during the course of infection. BALB/c mice were given 1 mg/ day of lovastatin beginning either: three days prior to RSV infection, one day prior to infection, one day post infection, or three days post infection. Untreated mice were also infected with RSV. Mice were bled 8 days post infection and serum cholesterol levels were measured using the ACE7 Cholesterol Reagent. Each group represents 3 mice and error bars represent standard deviation.

FIG. 5—Lovastatin eliminates RSV replication in cell culture. HEp-2 cell monolayers grown in 96 well plates were treated with either 10 μM lovastatin or left untreated beginning 24 hr prior to RSV infection (moi 0.1). Virus titer was measured for 8 consecutive days after RSV infection daily by plaque assay for eight days post RSV infection by harvesting the entire contents of a well and performing plaque assays in triplicate. RSV growth curve in untreated cells (□) and in cells treated with 10 μM lovastatin ( [symbol] ). Error bars represent standard deviation.

FIG. 6—Lovastatin diminishes cell-to-cell fusion. HEp-2 cells in 96 well plates were either treated with 20 μM lovastatin and infected with recombinant vaccinia virus expressing β-galactosidase gene under the direction of T7 polymerase or left untreated at the time of infection. Cell-to-cell fusion was determined by calculating the average number of blue cells. Error bars represent standard deviation calculated from the average number of fusion events in eight separate wells.

FIG. 7—Carboxy-terminal sequences of the known Rho family proteins.

FIG. 8—Cholesterol biosynthesis pathway with the formation of isoprenyl groups from mevalonate. The arrow at 1 indicates the rate-limiting step in cholesterol biosynthesis, which is catalyzed by the enzyme HMG-CoA reductase.

FIG. 9—Role of isoprenylation on RhoA partition to the cell membrane.

FIG. 10—RhoA inhibitors reduce HPIV-3 syncytia formation. HPIV-3-infected cells (multiplicity of infection (MOI) 0.1) showed extensive syncytia formation in HEp-2 cells two days post infection. Treatment with 5 μM GGTI-298, geranylgeranyltransferase inhibitor or 10 μM lovastatin beginning 24 hr prior to infection blocked HPIV-3-induced syncytia formation. Pretreatment of cells with 5 μM FTI-277, farnesyltransferase inhibitor, does not inhibit HPIV-3 syncytia formation. Addition of geranylgeranylpyrophosphate (GGP) or mevalonolactone to lovastatin treated cells rescues the inhibition by lovastatin. However, the addition of farnesylpyrophosphate (FP) to lovastatin treated cells does not rescue the inhibition by lovastatin.

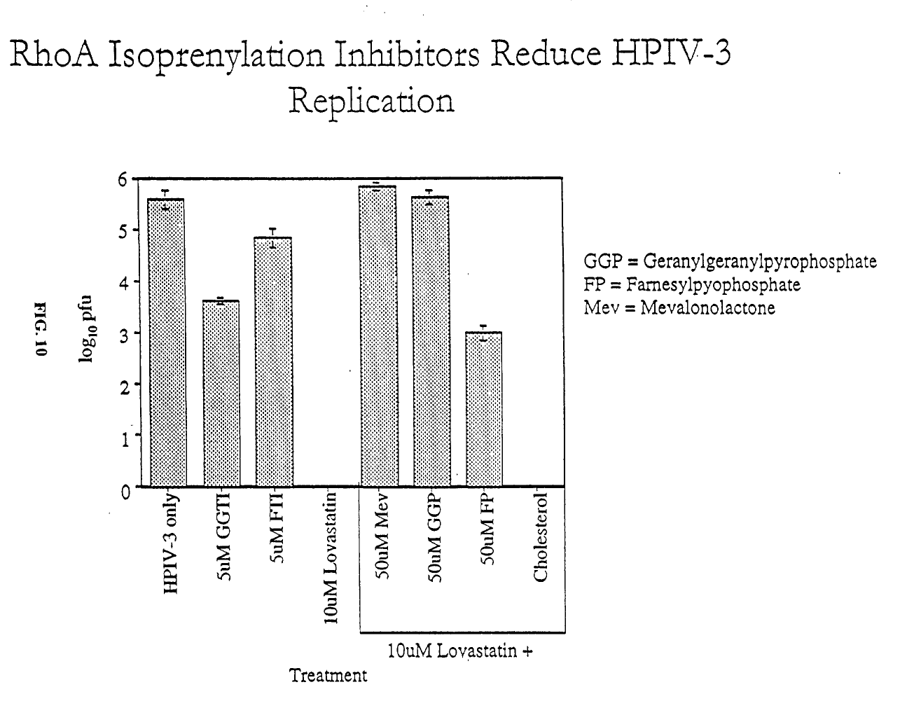

FIG. 11—RhoA inhibitors reduce HPIV-3 replication. HEp-2 cell monolayers grown in a 24-well plate were either treated with the RhoA inhibitors for 24 hrs or left untreated and infected with HPIV-3, MOI 0.1. Virus titer was measured 2 days after HPIV-3 infection by harvesting the entire contents of a well and performing plaque assays in triplicate. Treatment with 5μM GGTI-298 or the FTI-277 reduced HPIV-3 replication by 100-fold or 10-fold, respectively. 10 μM lovastatin beginning 24 hr prior to infection completely blocks HPIV-3 replication. However, the addition of 50 μM geranylgeranylpyrophosphate (GGP) or 50 μM mevalonolactone to these cells rescues the inhibition by lovastatin. The addition of 50 μM farnesylpyrophosphate (FP) to lovastatin treated cells partially restores HPIV-3 replication but the addition of cholesterol cannot rescue the inhibition by lovastatin.

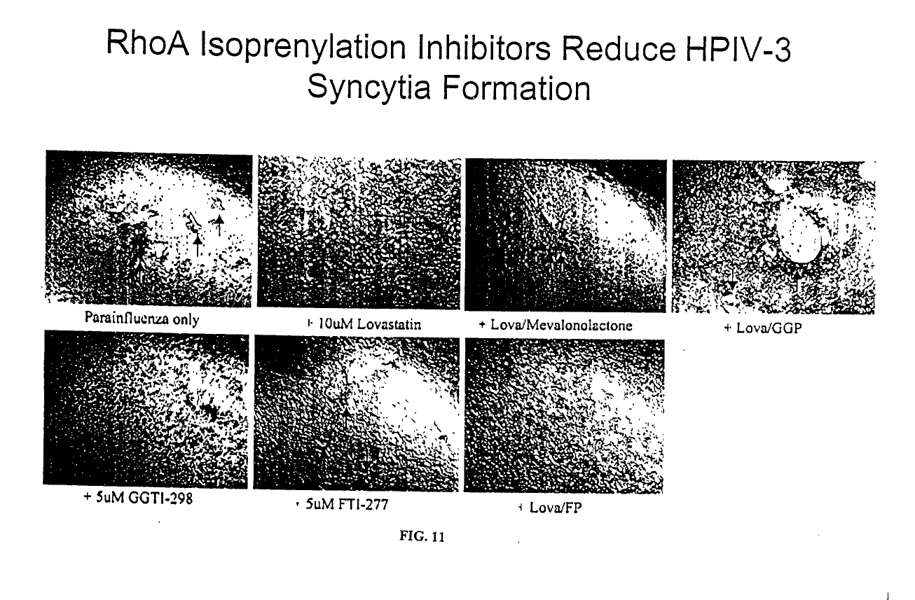

### DESCRIPTION OF ILLUSTRATIVE EMBODIMENTS

Previously, the present inventors showed that RhoA-derived peptides could be used to block viral infection of a susceptible cell. Their experiments revealed a role for Rho proteins, particularly RhoA, as mediators of cellular infection and cell-to-cell spread for viruses such as HIV-1, PIV-3, RSV, and others. The role of Rho proteins, particularly RhoA, and RhoA-derived peptides for the treatment of HIV, RSV, and other viral infections is described in copending U.S. Ser. No. 09/129,565 incorporated herein by reference.

Rho family proteins constitute one of three major branches of the Ras superfamily. Rho proteins share approximately 30 percent amino acid identity with the Ras proteins. At least 14 mammalian Rho family proteins have been identified thus far, including RhoA, RhoB, RhoC, RhoD, RhoE/Rnd3, Rnd1/Rho6, Rnd2/Rho7, RhoG, Rac1, Rac2, Rac3, Cdc42, TC10, and TTF.

G-proteins have been shown to undergo covalent processing at the carboxyl terminus, where a C-A-A-X amino acid motif is found. Processing includes lipidation, carboxymethylation and proteolytic removal of the last three amino acids. The lipid attached to certain G proteins has been identified as a C15 farnesyl isoprenoid attached by thioether linkage to the carboxy-terminal cysteine (Fukada et al., 1990; Lai et al., 1990). Mutation of the cysteine in the C-A-A-X domain prevents isoprenylation, thereby confirming that isoprenylation is required for intracellular transport and membrane attachment.

The use of 3-hydroxy-3-methylglutaryl-coenzymeA (HMG-CoA) reductase inhibitors has previously been described for the treatment of hypercholesterolemia. Maziere et al. (1994) also described a correlation between administration of the HMG-CoA reductase inhibitor lovastatin to the human H9 lymphocytic cell line and a decrease in reverse transcriptase activity in H9 cells infected with HIV- 1. This effect was postulated, however, to be the result of a change in the cholesterol/phospholipid ratio of the cell membrane, and the results were limited to early stages of infection in cultured cells.

HMG-CoA reductase inhibitors, like lovastatin, are currently used for treating hypercholesterolemia in humans (Alberts, 1988; Alberts et al., 1980; Parker et al., 1984). The inventors now report data that shows lovastatin can inhibit RSV replication in vitro and in vivo. Since lovastatin inhibits several pathways in the cell, such as the production of cholesterol and isoprenyl groups, there are several possible mechanisms by which lovastatin could inhibit RSV replication. Lovastatin may directly inhibit isoprenylation of RhoA, thereby preventing its location in the plasma membrane. Alternatively, it may lower the cholesterol content in cell membrane and alter lipid microdomains potentially interfering with either virus entry and membrane fusion or assembly and budding.

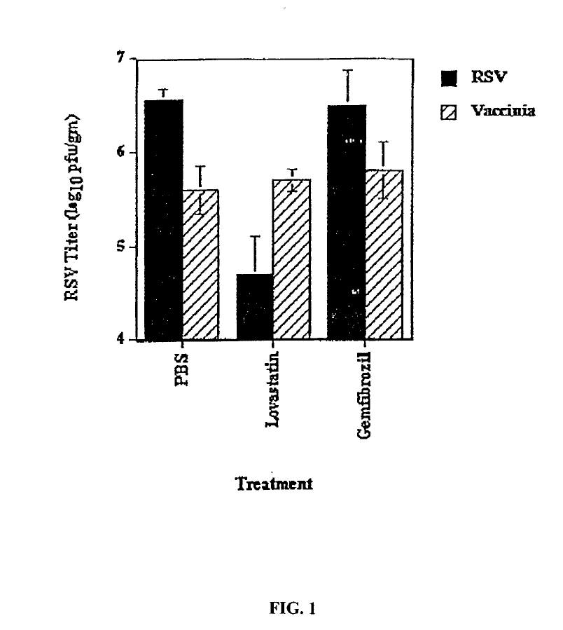

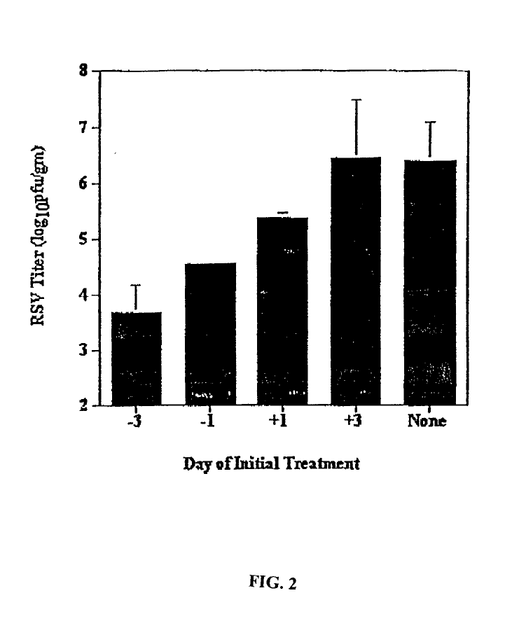

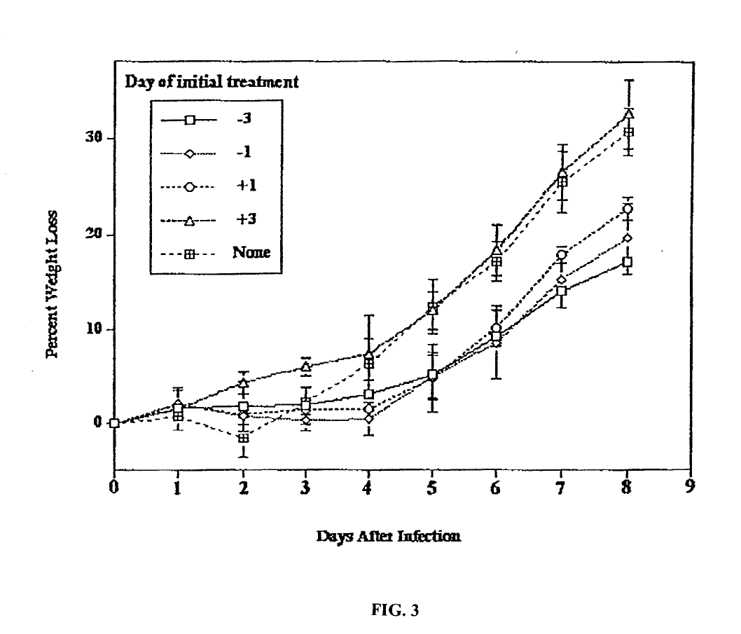

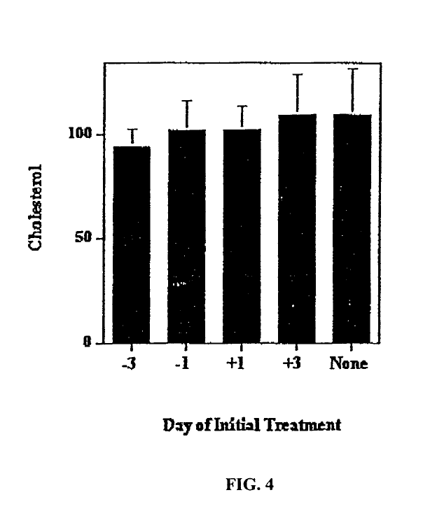

Regardless of the mechanism, the inventors show that mice treated with lovastatin beginning 3 days prior to infection have 100- to 1000-fold reduction of peak virus titers in lung 4 days after RSV infection (FIG. 1 and FIG. 2). In addition, lovastatin treatment reduces RSV-induced illness (data not shown) and weight loss (FIG. 3). Lovastatin decreased RSV replication and disease most effectively when given prior to infection or at very early stages of infection (FIG. 2 and FIG. 3). Since lovastatin does not inhibit vaccinia virus replication, and gemfibrozil (which also lowers cholesterol levels) does not inhibit RSV replication (FIG. 1), the inventors conclude that lovastatin is having a specific effect on RSV which is independent of cholesterol production. This is further indicated by data showing that mice treated with 1 mg/day of lovastatin for 11 days had no significant change in serum cholesterol levels (FIG. 4).

The implications of the inventors'findings are numerous, but in particular, indicate a new mode of treatment for viral infections that rely on interaction with RhoA or other isoprenylated small TPases. The details of the invention are provided below.

I. RhoA Receptors and Lipidation

RhoA is a member of the Rho family proteins, which constitute one of three major branches of the Ras superfamily. RhoA influences a variety of essential biological functions in eukaryotic cells including gene transcription, cell cycle, vesicular transport, adhesion, cell shape, fusion, and motility through its activation of signaling cascades (Takai et al., 1995). RhoA has also been shown to regulate smooth muscle contraction via Rho kinase (p160 ROCK) causing airway hyper-responsiveness.

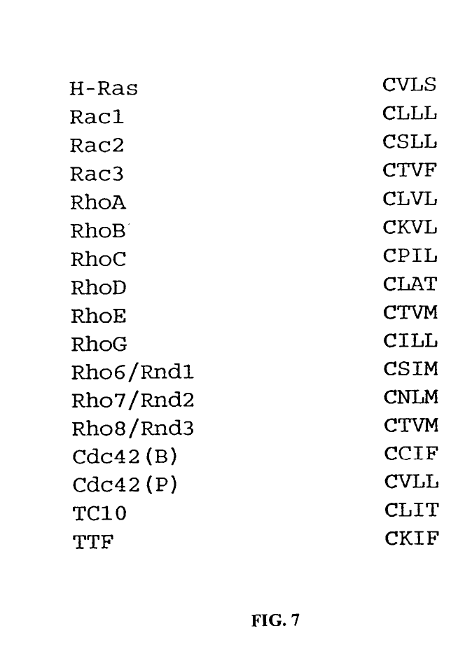

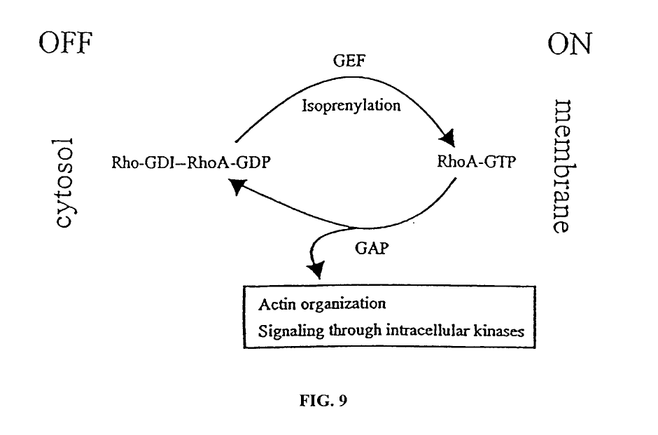

Rho proteins share approximately 30 percent amino acid identity with the Ras proteins. At least 14 mammalian Rho family proteins have been identified thus far, including RhoA, RhoB, RhoC, RhoD, RhoE/Rnd3, Rnd1/Rho6, Rnd2/Rho7, RhoG, Rac1, Rac2, Rac3, Cdc42, TC10, and TTF. These proteins have been shown to undergo covalent processing at the carboxyl terminus, where a C-A-A-X amino acid motif is found. FIG. 7. Processing includes lipidation, carboxymethylation and proteolytic removal of the last three amino acids. The lipid attached to certain G proteins has been identified as a C15 farnesyl isoprenoid attached by thioether linkage to the carboxy-terminal cysteine (Fukada et al., 1990; Lai et al., 1990). Mutation of the cysteine in the C-A-A-X domain prevents isoprenylation and membrane attachment. FIG. 9.

The determination of attachment of either the farnesyl group or the geranylgeranyl group on the cysteine residue appears to be related to the identity of the carboxy-terminal amino acid. For example, a leucine at the C-terminus has been shown to be correlated with geranylgeranylation (Moores et al., 1991), while the presence of a methionine, serine, or phenylalanine residue increases affinity of binding to the rat brain p21 ras farnesyl transferase (Reiss et al., 1991). As indicated in FIG. 7, the carboxy-terminal residue in RhoA is a leucine, which directs the addition of a geranylgeranyl group to the cysteine residue. However, RohB, which has sequence homology with RhoA in the region of fusion protein binding, has a carboxy-terminal methionine.

Farnesylation depends upon the attachment of a 15-carbon isoprenoid moiety to the carboxy-terminal cysteine, catalyzed by the enzyme farnesyltransferase (FT). Geranylgeranylation depends upon the attachment of a 20-carbon isoprenoid moiety, catalyzed by the enzymes geranylgeranyl transferase type I (GGT-I) and geranylgeranyl transferase type II (GGT-II). Farnesyl transferase (FT) and GGT-I have been shown to modify Ras superfamily proteins that have the C-A-A-X motif, while GGT-II mediates geranylgeranylation of non-CAAX-containing proteins.

II. Target Viruses

The present invention provides for prophylactic and therapeutic intervention against viral infections. In particular, the viruses that utilize the RhoA receptor as part of their infectious process will be inhibited by the administration of agents that inhibit to availability of RhoA to such invading agents. Below is a non-limiting list of such viruses.

A. Human Respiratory Syncytial Virus

Human respiratory syncytial virus is a pneumovirus in the family Paramyxoviridae. It is a non-segmented negative-strand RNA virus, with a cytoplasmic replication program. The viral nucleocapsid is packaged in a lipoprotein envelope that is acquired from the host cell plasma membrane during budding. The virus has a fusion protein (RSV F) and a G glycoprotein (RSV G). RSV can infect cells as a cell-free virus, but can also spread by syncytium formation between infected cells and uninfected neighboring cells. Membrane fusion is important for both virus entry and for cell-to-cell spread.

RSV is the leading viral cause of severe lower respiratory tract illness in infants and young children (Walsh and Graham, 1999). RSV can also cause severe illness and death in the elderly (Treanor and Falsey, 1999) and immunocompromised bone marrow (Hertz et al., 1989; Wendt et al., 1995) and lung transplant patients (Wendt et al., 1995). The mortality rate in bone marrow transplant patients is between 70 and 100% (Hertz et al., 1989).

Although RSV-induced disease in infants may be primarily immune-mediated, in bone marrow and lung transplant recipients and in persons with severe combined immunodeficiency syndrome the pathology, characterized by giant cell formation, is related to ongoing viral replication. In addition, infants with AIDS have been shown to have continuous viral shedding for over 200 days (King et al., 1993). These patient groups would benefit from more effective antiviral therapeutic options for RSV. It is more likely that antiviral prophylaxis would be required to make an impact on illness in infants and the elderly.

RSV G is thought to be the attachment glycoprotein of RSV, although the host cell receptor has never been identified. A cold-passaged B-strain RSV has been shown to infect cells having a deleted G, indicating that other RSV proteins may be sufficient for RSV attachment. One of these other proteins, RSV F, is thought to be trimeric with three transmembrane virion proteins assembled separately to make up the membrane spikes. RSV G and the small hydrophobic protein (RSV SH) may also be part of the membrane spike structure. The F protein contains a cleaved N terminal signal sequence. The protein requires endoproteolytic cleavage into F1 and F2 to be functional. Results of recent experiments, indicating that whole RhoA enhances viral infection of cells, but that RhoA peptide from the fusion protein binding region inhibits viral infection, suggest that RhoA may be a host cell RSV receptor or coreceptor. These results are described in copending U.S. Ser. No. 09/129,565.

The present inventors have shown that RhoA is activated by RSV infection and inactivating RhoA with C3 toxin from Clostridium botulinum, which ADP ribosylates RhoA on Asn 41, prevents RSV-induced syncytia formation (Stein et al., 1999; Treanor and Falsey, 1999). Therefore, RhoA-mediated signaling may play a role in various aspects of RSV pathogenesis including cell-to-cell fusion, secretion of IL-1β, IL-6 and IL-8, and airway hyper-responsiveness. In any event, its identification as a receptor for RSV renders this virus susceptible to therapeutic attack at the level of the RhoA receptor.

Rodent models of RSV have been used to study pathogenesis and to evaluate candidate vaccines and anti-viral agents including passive antibody approaches. The mouse and cotton rat have been especially accurate in predicting the clinical response to anti-viral agents active against RSV. The only licensed product for treating severe RSV infection is ribavirin, which has been demonstrated in rodent models (Wyde et al., 1987; Gruber et al., 1987). Two other antibody products, one polyclonal and one monoclonal have been licensed for prophylactic treatment of RSV in selected risk groups. Our laboratory was involved in evaluating those products in preclinical studies in the mouse model, and our results accurately reflected the behavior of these products in subsequent clinical trials (Graham et al., 1993, 1995; Fisher et al., 1999). The assessment of RSV anti-viral agents in African Green Monkeys or Chimpanzees is prohibitively expensive, and no more predictive than the rodent models for testing the anti-viral effects of agents active against RSV.

B. Human Parainfluenza Virus

Human parainfluenza virus types 1 (PIV-1), 2 (PIV-2), and 3 (PIV-3) are members of the Paramyxoviridae family which also cause serious lower respiratory tract disease in infants and children. These infections account for approximately 18% of all hospitalizations of pediatric patients for respiratory tract infection (Collins et al., 1996; Marx et al., 1997). A vaccine has not been approved for the prevention of parainfluenza infection, and there is no truly effective antiviral therapy once disease is established.

Parainfluenza viruses also express a fusion glycoprotein with a similar binding sequence to that of RSV. In fact, in results of previous experiments by the inventors, parainfluenza virus infection of normally susceptible cells was demonstrated to be mediated by RhoA. Thus, like RSV, PIV is susceptible to treatment with both HMG-CoA reductase inhibitors and inhibitors of isoprenylation.

C. Human Immunodeficiency Virus

HIV-1 is a lentivirus with a viral fusion glycoprotein, gp41, which has also been demonstrated to interact with RhoA. Addition of a peptide comprising the sequence of amino acids 77-95 (RhoA₇₇₋₉₅) was shown to inhibit both infection and syncytia formation by HIV in cultured MT-2 cells in experiments reported by the inventors in U.S. Ser. No. 09/129,565.

Although tremendous advances have been made in the treatment of HIV type 1 (HIV-1) infection, the newer anti-HIV-1 regimens do not fully eliminate the virus infection, thereby requiring lifelong treatment to postpone the multitude of symptoms of acquired immunodeficiency syndrome (AIDS). Many of the newer therapies are also difficult to administer in the developing countries, where HIV-1 infection exacts its greatest toll.

HIV successfully evades many treatment regimens because drug-resistant viruses are so common. New virions can be synthesized at a rate of as many as 10¹⁰ virions per day. Productively infected CD4+ cells survive only 2.2 days, being rapidly replaced and maintaining a somewhat constant population of infected cells. The large population size, combined with the fact that HIV replication is highly error-prone (reverse transcriptase, for example, makes about one error per 10 bases copied), results in an evolutionary “factory” for drug-insensitive mutant viruses.

The protease inhibitors have provided one method of treatment for HIV infection. HIV proteins must be cleaved at the appropriate sites late in the viral replication cycle in order to form the components of new infectious viral particles. Protease inhibitors block this cleavage, limiting the number of viral particles that can be formed. However, HIV mutants resistant to protease inhibitors can also arise resulting in relapse in a patient undergoing treatment.

Mixtures of antiviral drugs have also shown some promise. Triple combination therapy, for example, combines cocktails of compounds with different modes of action. A common cocktail consists of two reverse transcriptase (RT) inhibitors and a protease inhibitor. Unfortunately, however, even with triple combination therapy, studies have reported that patients in whom viral loads have been decreased still harbor replication-competent virus in some CD4+ resting T cells (Finzi, 1997; Wong, 1997; Chun, 1997). Furthermore, many patients cannot tolerate the side effects of triple combination therapy.

Early efforts to treat and prevent HIV infection were directed to the interaction of HIV-1 with its cellular receptor. The first event in viral infection is recognition of surface receptors on the host cell. For HIV, a number of cell surface fusion receptors have been found. Most prominent among these is the HLA class II receptor, CD4. CD4-related viral entry is initiated by a high-affinity interaction between CD4 and the surface glycoprotein (gp120) of the virus. Conformational. changes then occur within the viral transmembrane protein gp41, leading to fusion of the viral membrane with the cellular membrane.

Several members of the family of seven-transmembrane G-protein-coupled proteins, particularly chemokine coreceptors, have also been identified as cofactors for HIV-1 fusion. CCR5 has been shown to mediate fusion of viruses characterized as macrophage tropic or dual tropic (Alkhatib et al., 1996), while CXCR4 has been demonstrated to mediate fusion of T-cell-tropic or dual-tropic strains (Endres et al., 1996). Bandres et al. (1998) have recently shown that the HIV envelope (Env) interacts with CXCR4 independently of CD4 but that this binding is enhanced by prior interaction of Env with soluble CD4, indicating that the interaction of both receptors with HIV enhances binding. Other coreceptors include CCR3 (Choe et al., 1996) and CCR2b (Doranz et al., 1996). The discovery of multiple receptors, and the later indication that HIV binding, although possibly independent in terms of the virus and each receptor, was enhanced by viral binding to multiple receptors, has made it more difficult to target the cell surface fusion receptors for inhibition of HIV infection.

D. Ebola Virus

Ebola virus outbreaks result in extremely high mortality rates. Ebola fusion protein binds to RhoA in a yeast two-hybrid system. Thus, Ebola should also be susceptible to inhibition according to the present invention.

E. Non-Human Viruses

Bovine respiratory syncytial virus (BRSV) is a common agent that has been associated with pre-weaning pneumonia in calves. While both modified live and killed BRSV vaccines are available, control of BRSV spread from infected calves to other unvaccinated calves can be accomplished by administration of HMG-CoA reductase inhibitors to both infected and uninfected calves to inhibit viral infection of susceptible cells. Adjustment of the doses previous described for veterinary purposes is well within the skill of a licensed veterinarian, and will depend upon the general health of the animal, its size, and its age.

Other non-human viruses that should be targets for the therapies claimed herein including Newcastle's Disease virus, turkey rhinotracheitis and canine distemper.

III. Inhibitors of HMG-CoA Reductase

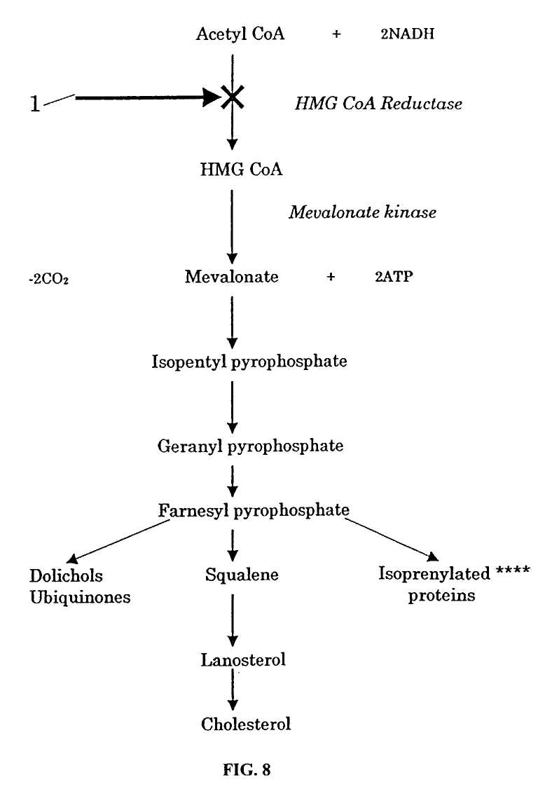

HMG-CoA reductase catalyzes the conversion of 3-hydroxy-3-methylglutarylcoenzyme A to mevalonate, a cholesterol precursor. Mevalonate, however, is also a precursor for the 15-carbon farnesyl pyrophosphate and the 20-carbon geranylgeranyl pyrophosphate, as shown in FIG. 8. Therefore, by inhibiting the formation of mevalonate, production of the isoprenyl group required for cell membrane expression of RhoA is also inhibited.

Production and use of HMG-CoA reductase inhibitors has been described previously, and at least five HMG-CoA reductase inhibitors are currently on the commercial market. These include lovastatin (Mevacor(R), Merck & Co., Inc.), pravastatin (Pravachol, E.R. Squibb & Sons, Inc.), simvastatin (Zocor, Merck & Co., Inc.), fluvastatin (Lescol, Novartis), atorvastatin (Lipitor, WarnerLambert) and mevastatin (Compactin).

Lovastatin is an FDA-approved drug that is used to treat hypercholesterolemia. It inhibits HMG-CoA reductase, an important enzyme in the cholesterol biosynthesis pathway (Alberts, 1988; Alberts et al., 1980; Endo et al., 1976; MacDonald et al., 1988; Parker et al., 1984). Lovastatin is also used to study isoprenylation and membrane localization of proteins such as RhoA, since a branch of the cholesterol biosynthesis pathway leads to the formation of isoprenyl groups (Koch et al., 1997; Park and Galper, 1999). Lovastatin inhibits the production of geranyl pyrophosphate and farnesyl pyrophosphate and therefore inhibits protein isoprenylation (Alberts, 1988; Alberts et al., 1980).

In immunocompromised patients, lower doses of HMG-CoA reductase inhibitors may be indicated. Generally, however, the method of the present invention describes the use of lovastatin at doses of from 10 to 160 mg per day, which can be achieved by administration of commercially available tablets of 10, 20, or 40 milligrams. Where pravastatin is the drug of choice, doses are described between 10 to 40 milligrams daily, also achieved by administration of commercially available tablets of 10, 20, or 40 milligrams. Alternately, simvastatin may be used to block mevalonate synthesis. Daily doses of simvastatin range between 5 to 80 milligrams, with tablets of 5, 10, 20, 40 and 80 milligrams commercially available. Fluvastatin or atorvastatin may also be used to inhibit the formation of the isoprenyl group required for RhoA modification for cell surface expression. Fluvastatin doses range between 20 to 40 milligrams, administered as 20 or 40 milligram tablets. Atorvastatin does range between 10 to 80 milligrams, administered as 10, 20, or 40 milligram tablets.

Patients for whom HMG-CoA reductase inhibitors are indicated include virus-infected individuals who have not yet begun a treatment regimen, virus-infected individuals for whom other treatment regimens have already failed to reduce viral spread, and patients for whom a combination therapy may be more effective.

IV. Inhibitors of Isoprenylation

As stated previously, RhoA is one of many cellular proteins which require modification by either geranylgeranyl transferase or farnesyl transferase for partition to the cell membrane. RhoA in particular requires the addition of a geranylgeranyl moiety to the carboxy-terminal cysteine for cell membrane attachment. Therefore, by inhibiting the transfer of the geranylgeranyl moiety to the C-terminal cysteine, cell surface expression of the RhoA molecule also can be inhibited. Direct inhibition of the prenyl transferase has fewer potential side effects than inhibition of formation of the isoprenyl groups by HMG-CoA reductase inhibition of cholesterol biosynthesis.

Inhibitors of geranylgeranyl-protein transferase (GGT) have been described in U.S. Pat. No. 5,470,832 (Gibbs & Graham). These compounds can be administered to an individual infected with a virus in dosage amounts of between 0.5 mg/kg of body weight to about 20 mg/kg of body weight. In those individuals in whom infection has not yet produced an immunocompromised state, dosage amounts should be in the upper end of the stated range. Specific doses are determined on a case-by-case basis by the individual patient's treating physician, taking into account the patient's general health condition, size, and age. Dosage calculations are commonly adjusted to compensate for these factors, and methods of doing so are well known to those of skill in the art.

Combination of geranylgeranyl transferase inhibitors with farnesyl transferase inhibitors is another method of treatment for virus infection since cross-specificity of the C-A-A-X box protein prenyl transferases has been reported (Trueblood et al., 1993). In the presence of farnesyl protein transferase inhibitors, for example, Ras proteins in the human colon carcinoma cell line DLD-1 were shown to be alternatively prenylated by geranylgeranyl transferase-I (Whyte et al., 1997). Therefore, in order to ensure inhibition of RhoA partition to the cell surface, a combination of one or both a geranylgeranyl transferase inhibitor and a farnesyl transferase inhibitor can be utilized.

Farnesyl transferase inhibitors generally fall into two classes: analogs of farnesyl diphosphate; and protein substrates for farnesyl transferase. Farnesyl transferase inhibitors have been described in U.S. Pat. No. 5,756,528, U.S. Pat. No. 5,141,851, U.S. Pat. No. 5,817,678, U.S. Pat. No. 5,830,868, U.S. Pat. No. 5,834,434, and U.S. Pat. No. 5,773,455, incorporated herein by reference. Among the farnesyl transferase inhibitors shown to be effective for inhibiting the transfer of the farnesyl moiety to Ras-related proteins are L-739,749 (a peptidomimetic analog of the C-A-A-X sequence), L-744,832 (a peptidomimetic analog of the C-A-A-X sequence), SCH 44342 (1-(4-pyridylacetyl)-4-(8-chloro-5,6 dihydro-IIH benzo [5,6] cyclohepta [1,2-b]pyridin-11-yhdene)piperidine), BZA-5B (a benzodiazepine peptidomimetic), FTI-276 (a C-A-A-X peptidomimetic), and B1086 (a C-A-A-X peptidomimetic). Administration of farnesyl transferase inhibitors (FTIs) is accomplished by standard methods known to those of skill in the art, most preferably by administration of tablets containing the FTI, and is expected to fall approximately within a range of about 0.1 mg/kg of body to weight to about 20 mg/kg of body weight per day.

A method of treating transplant patients and other individuals at risk of infection by RSV and parainfluenza is administration of one or more geranylgeranyl transferase inhibitors and/or farnesyl transferase inhibitors. Dosages for immunocompromised individuals are more appropriately within the lower end of the range of dosages for geranylgeranyl transferase inhibitors and farnesyl transferase inhibitors previously described. In some individuals, a preferred method of administration may be intravenous, while in other individuals administration of tablets by oral route is indicated. Individual doses can be determined by the patient's physician, taking into account the health of the patient, the age of the patient, and the size of the patient, as is customarily done by treating physicians for most medications.

Geranylgeranyl transferase inhibitors and farnesyl transferase inhibitors can also be used to inhibit viral spread and treat disease caused by other viruses which use RhoA to mediate infection and cell-to-cell spread. These viruses can be identified by their binding affinity to the RhoA viral binding moiety, identified in U.S. Ser. No. 09/129,565. Methods of determining viral affinity to cellular receptors are known to those of skill in the art, and such a determination can be made using standard techniques, to assess viral binding affinity to RhoA. For highly infectious viruses, for example, isoprenylation inhibitors can be used prophylactically (in doses in the upper range of those previously described for each type of inhibitor) for individuals at risk of infection, such as health care workers and school personnel.

It will be appreciated by one of skill in the art that, as used herein, inhibitors of RhoA isoprenylation are those compounds which either directly or indirectly inhibit the addition of a farnesyl or geranylgeranyl moiety to the carboxy-terminal cysteine residue of the C-A-A-X motif of the RhoA molecule. Direct inhibitors, for example, include inhibitors of farnesyl transferase and geranylgeranyl transferase. Indirect inhibitors, for example, can include inhibitors of HMG-CoA reductase, since HMG-CoA reductase enzymatically mediates the reaction which results in the formation of mevalonate, a precursor for farnesyl pyrophosphate and geranylgeranyl pyrophosphate. Therefore, isoprenylation inhibitors can include any compound which inhibits either the formation of a precursor molecule needed for the formation of the farnesyl or geranylgeranyl moiety, a direct inhibitor of the formation of the farnesyl or geranylgeranyl moiety, or an inhibitor of one of the enzymes required for the transfer of the farnesyl or geranylgeranyl moiety to the CA-A-X motif during isoprenylation.

V. Combination Therapies

Virus resistance to traditional therapies represents a major problem in clinical virology. Thus, one goal of current research is to find ways to improve the efficacy of traditional therapies, as well as decrease the emergence of virus resistance. Thus, in the context of the present invention, it is contemplated that inhibitors of HMG-CoA reductase and inhibitors of isoprenylation may be used as combination therapies with each other, and with other more traditional therapies.

For example, for RSV infections, one may utilize an monoclonal or polyclonal antibody composition that binding immunologically to an RSV determinant. Alternatively, ribavarin (Virazole) may be employed. For HIV, one may utilize a nucleoside analog, such as AZT, and non-nucleotide reverse transcriptase inhibitor, an integrase inhibitor, a protease inhibitor, or an inhibitor of virus entry, such as T-20.

To inhibit virus infection, using the methods and compositions of the present invention, one will treat a patient with an inhibitor of the present invention and a traditional antiviral therapeutic. This process may involve administration of both therapies at the same time, for example, by administration of a single composition or pharmacological formulation that includes both agents, or by administering to said patient two distinct compositions or formulations, at the same time.

Alternatively, the traditional therapy may precede or follow the present inhibitor treatment by intervals ranging from minutes to weeks. It is also conceivable that more than one administration of either treatment will be desired. Various combinations may be employed, where the inhibitor is “A” and the traditional therapeutic is “B”:

A/B/A B/A/B B/B/A A/A/B A/B/B B/A/A B/B/B/A B/B/A/B
A/A/B/B A/B/A/B A/B/B/A B/B/A/A B/A/B/A B/A/A/B
A/A/A/B B/A/A/A A/B/A/A A/A/B/A A/B/B/B B/A/B/B

Similarly, the present invention may comprise a combination of an HMG CoA reductase inhibitor (“A”) with an inhibitor of geranylgeranyl transferase or farnesyl transferase

VI. Pharmaceutical Formulations and Routes of Administration

Acceptable doses of HMG-CoA reductase inhibitors, farnesyl transferase inhibitors and geranylgeranyl transferase inhibitors, for example, are described herein. In hands of a physician skilled in the art of prevention and treatment of viral infection, these compounds can be administered to any patient at risk of infection by a virus which requires RhoA as a mediator of infection. Appropriate doses for the prevention of viral infection, or the treatment of cell-to-cell spread of virus once infection has been established, can be determined by the treating physician, taking into consideration the general health, age, and size of the subject.

Methods of administration of HMG-CoA reductase inhibitors and isoprenylation inhibitors are oral administration of tablets, caplets, or capsules, and oral administration of liquid. Alternate methods of administration may include, for example, enteral, parenteral, intravenous, or by nasogastric tube. Solid and aqueous compositions of the present invention will have an effective amount of an inhibitor that decreases the ability of a virus to infect target cells, either alone or in combination with another agent. Such compositions will generally be dissolved or dispersed in a pharmaceutically acceptable carrier or aqueous medium. “Pharmaceutically or pharmacologically acceptable” refers to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

The inhibitors of the present invention will often be formulated for parenteral administration, e.g., formulated for injection via the intravenous, intramuscular, subcutaneous, or even intraperitoneal routes. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. The form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, perfusion over a period of time via catheterization, and even time-release capsules and tablets.

VII. Methods of Treating and Preventing Viral Infections

The inventors previously have indicated that RhoA is a mediator of viral infection for a number of viruses, including HIV, RSV and PIV-3. Both RSV and parainfluenza have been associated with serious, and often fatal, disease in transplant recipients—particularly bone marrow recipients, and HIV is a health risk of world-wide proportions. Thus, in one embodiment of the present invention, one or more HMG-CoA reductase inhibitors or isoprenylation inhibitors are administered to a subject infected with or at risk of infection with HIV, RSV or PIV-3. The choice of an inhibitory compound will be determined by the patient's treating physician, and may include, but not be limited to, the HMG-CoA reductase inhibitors (lovastatin, pravastatin, simvastatin, fluvastatin, and atorvastatin) and isoprenylation inhibitors.

In one embodiment of the present invention, the patient at risk of viral infection is a transplant recipient. Since transplant recipients are immunocompromised individuals, lower doses of inhibitors are indicated. It is well known to those in the medical profession that individual dosage of medication can be varied according to the current state of the patient's health, the size of the patient, and the patient's age. Therefore, individual dosage will be most effectively determined by the patient's treating physician, providing a maximum dose indicated for the individual patient.

Infants and young children who suffer from severe combined immunodeficiency syndrome (SCIDS), as well as other immunocompromized individuals, such as patients receiving bone marrow or lung transplant, or undergoing other solid organ transplant or chemotherapy. can also benefit from the method of the present invention. In these individuals, inhibitors of HMG-CoA reductase and isoprenylation can be used prophylactically to prevent infection after suspected exposure to an infectious virus or for therapy to prevent the spread of infectious virus within the body. RSV/PIV combination infections are not uncommon in SCIDS patients (McIntosh et al., 1984). Since both of these viruses have been shown to require RhoA for the establishment of infection, the method of the present invention provides a means of protecting against RSV/PIV infection in immunocompromised patients, as well as treating established infection by inhibiting cell-to-cell spread of both viruses.

VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Virus and Cells. The A2 strain of RSV was provided by Dr. R. Chanock, NIH, Bethesda, Md. RSV stocks were prepared as previously described (Graham et al., 1988). HEp-2 cells were maintained in Eagle's minimal essential media supplemented with glutamine, gentamicin, penicillin G, and 10% fetal bovine serum (FBS).

Plaque assay. Two-day-old HEp-2 monolayers, 80% confluent in 12-well plates (Costar, Cambridge, Mass.) were used for plaque assay. The assay was done as previously described (Graham et al., 1988).

Mouse studies. A dose response curve from 0.5 mg/day to 20 mg/day lovastatin was performed on C57BL6 mice to determine the optimal concentration for inhibition of RSV. 1 mg/day and 5 mg/day inhibited RSV replication equivalently, therefore the inventors chose to continue the studies using 1 mg/day lovastatin. The dose for gemfibrozil (50 mg/day) was chosen because, relative to doses recommended for humans, it is an equivalent dose to 1 mg/day lovastatin in humans. Eight-week old female pathogen free C57/BL6 or BALB/c mice (Harlan-Sprague Dawley, Indianapolis, Ind.) were given 1 mg/day lovastatin (Merck, N.J.) in 100 μl PBS, 100 μl PBS per day, or 50 mg/day gemfibrozil (UDL, Rockford, Ill.) in 200 μl by PBS by oral gavage starting at various times prior to and after virus infection and throughout the course of the study. Mice were anaesthetized and infected intranasally with 1×10⁶ pfu RSV or 1×10⁵ vaccinia virus. Lungs were harvested for RSV and vaccinia plaques assays as described (Graham et al., 1988) four days post RSV infection.

Cell fusion assay using vaccinia virus-based expression of RSV envelope glycoproteins. The ability of lovastatin to inhibit RSV induced cell-to-cell fusion was assessed using a fusion assay. One population of HEp-2 cells was infected with recombinant vaccinia virus vTF7 3, which encodes T7 polymerase, at a multiplicity of infection of 10 PFU per cell, and was transfected with plasmids encoding RSV glycoproteins F, G and SH under control of the T7 promoter (gifts from P. Collins, National Institutes of Health, Bethesda, Md.) using FuGene (Boehringer Mannheim, Indianapolis, Ind.). At 5 hr after transfection, the cells expressing viral envelope proteins were trypsinized, suspended in MEM containing 2.5% FBS to a density of 2×10⁷ cells per ml, and incubated overnight at 32° C. The cells were then washed and suspended in

OptiMEM (Gibco BRL, Grand Island, N.Y.) at a concentration of 1×10⁶ cells per ml. A second population of HEp-2 cells was infected with recombinant vaccinia virus expressing β-galactosidase under control of the T7 promoter (provided by E. A. Berger, National Institutes of Health, Bethesda, Md.). The cell population infected with recombinant vaccinia expressing β-galactosidase was split in half. Half of the cells were left untreated and the other half was treated with 20μM lovastatin for 24 hr beginning at the time of infection. At 5 hr after infection, cells were trypsinized and finally suspended at a concentration of 1×10⁵ cells per ml. The two cell populations were mixed in triplicate by adding 100 μl of each cell population to 96-well tissue culture plates, which were then incubated at 37° C. for 4 hr. At 4 hr the cells were fixed in 2% glutaraldehyde/20% formaldehyde (Sigma, St Louis, Mo.) in PBS for 10 min. 150 μl X-gal solution (1M potassium ferricyanide, 1M potassium ferrocyanide, 1M magnesium chloride, and X-gal (Fisher, Springfield, N.J.)) was added. After 8 hr, blue-stained fused cells were viewed with an inverted phase contrast microscope.

### Example 2

### Results

Lovastatin diminishes RSV replication in mice. To determine if lovastatin could inhibit RSV replication in vivo, mice were treated with 1 mg/day lovastatin, 50 mg/day gemfibrozil, or PBS by oral gavage beginning three days prior to infection with either RSV or vaccinia virus. Vaccinia replication (FIG. 1) and illness was not effected by lovastatin or gemfibrozil treatment compared to PBS treated controls. Gemfibrozil and PBS treated mice infected with RSV had a peak titer in the lung of 6.5+/−0.43(log₁₀ pfu/gm) and 6.5+/−0.19(log₁₀ pfu/gm), respectively, while RSV replication in lovastatin treated mice was reduced by nearly 100-fold to 4.7+/−0.4 (log₁₀ pfu/gm).

To determine if lovastatin could effectively inhibit virus replication if given after infection, mice were treated with 1 mg/day lovastatin beginning either 3 days prior to infection, 1 day prior to infection, 1 day after infection, or 3 days post infection (FIG. 2). Untreated mice and mice given lovastatin starting 3 days after RSV infection had similar viral titers in the lung on day 4 of 6.2+/−0.79 (log₁₀ pfu/gm) and 6.3+/−1.1 (log₁₀ pfu/gm), respectively (FIG. 2). Mice treated with lovastatin beginning 3 days prior to infection had more than a 100-fold reduction in viral titer to 3.8+/−0.48 (log₁₀ pfu/gm) compared to untreated mice (FIG. 2). Lovastatin was also able to reduce RSV replication when mice were treated soon after infection. Mice treated beginning 1 day after RSV infection had viral titers reduced by 10-fold. However, mice treated with lovastatin beginning 3 days after infection had no reduction in viral titers compared to untreated mice.

Lovastatin diminishes RSV-induced illness in mice. Mice were also weighed daily and given illness scores as a measure of illness. Mice treated with lovastatin 3 days after RSV infection had a similar weight loss curve (FIG. 3) and illness scores compared to untreated RSV-infected mice. Both groups had a peak percent weight loss of about 30% at 8 days post infection (FIG. 3). Mice treated with lovastatin at earlier time points in the infection had reduced illness. Peak weight loss 8 days post infection in mice treated with lovastatin 3 days prior to infection, 1 day prior to infection, and 1 day post infection was 17%, 19%, and 22%, respectively (FIG. 3). Therefore, the degree of inhibition by lovastatin was dependent on the time treatment was started. RSV-induced illness was also diminished in lovastatin-treated mice compared to untreated mice or gemfibrozil treated mice as measured by percent weight loss. Mice treated with PBS, gemfibrozil, and lovastatin had peak percent weight loss on day 8 post infection of 27%, 40%, and 19% respectively. Uninfected mice treated with either 1 mg/day lovastatin or 50 mg/day gemfibrozil for 11 days had no significant weight loss.

Lovastatin does not affect serum cholesterol during acute infection. Since, lovastatin reduces total serum cholesterol levels over time, the inventors wanted to determine if this could be the cause of reduced RSV replication in mice. To determine whether lovastatin could reduce serum cholesterol levels in the time frame of this study, serum samples were collected from mice treated 3 days before infection, 1 day before infection, 1 day after infection, 3 days after infection, and untreated mice 8 days post RSV infection. Serum cholesterol levels were measured using the ACE7 Cholesterol Reagent. There were not significant differences in serum cholesterol levels between groups (FIG. 4).

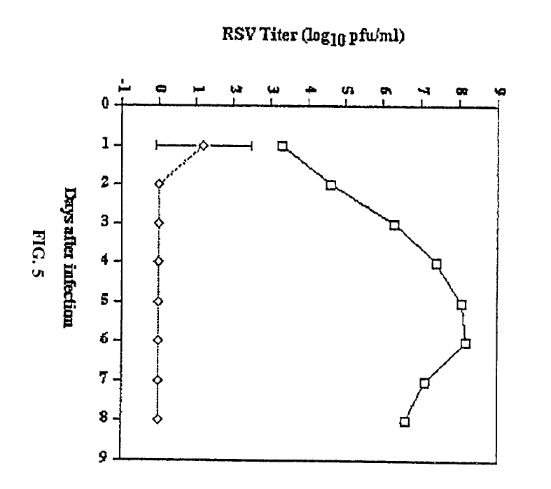

Lovastatin eliminates RSV replication in HEp-2 cells. Next, the inventors asked whether lovastatin could alter RSV replication in cell culture. HEp-2 cells in a 96-well plate were treated with either 10 μM lovastatin or left untreated beginning 24 hr prior to RSV infection. The contents of an individual well were transferred to HEp-2 monolayers in twelve-well plates for plaque assay for eight consecutive days after RSV infection (FIG. 5). RSV replicates normally in untreated HEp-2 cells with viral replication peaking on days 4-6 post RSV infection. Interestingly, RSV replication is completely inhibited in lovastatin-treated cells. This defect can be rescued in cells that have been treated with 10 μM lovastatin for 24 hr followed by treatment with 20 μM mevalonolactone which rescues the cholesterol biosynthetic pathway just downstream of HMG-CoA reductase. This indicates the lovastatin effect on RSV replication is mediated by the products of this biosynthetic pathway and not by alternative mechanisms.

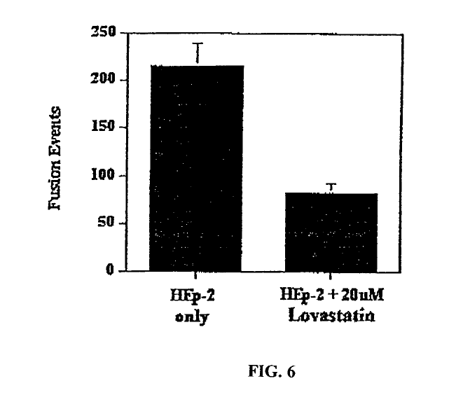

Lovastatin diminishes cell-to-cell fusion. Next, the inventors asked whether lovastatin can inhibit RSV mediated cell-to-cell fusion. The inventors used a fusion assay in which one set of HEp-2 cells transfected with RSV envelope proteins, F, G, and SH and infected with a recombinant vaccinia virus expressing T7 polymerase. Another set of HEp-2 cells was infected with a recombinant vaccinia virus encoding a lacZ gene under the direction of a T7 promoter. One half of the cells containing the lacZ gene were treated with 20 μM lovastatin at the time of vaccinia infection. The other half was left untreated. After mixing the two cell populations for 4 hr, cells were fixed and x-gal was added. The number of blue cells in the lovastatin-treated cells and the untreated cells was counted. Untreated cells had an average of 215+/−29 fusion events per well while lovastatin-treated target cells had an average of 75+/−11 fusion events per well (FIG. 6). Therefore, lovastatin can diminish virus-induced cell-to-cell fusion by greater than 50% when cells are treated 16 hr prior to mixing the two cell populations. This indicates that RhoA localization in the plasma membrane may be important for RSV-mediated cell-to-cell fusion.

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Adamson, Marshall, Hall, Tilbrook, “Post-translational modifications of p21rho proteins,” J. Biol. Chem., 272: 20033-20038, 1992.

Alberts, “HMG-CoA reductase inhibitors - the development,” In: Atherosclerosis Reviews, New York Raven Press, 18:123, 1988.

Alberts, Chen, Kuron, Hunt, Huff, Hoffman, Rothrock, Lopez, Joshua, Harris, Patchett, Monaghan, Currie, Stapley, Albers, Schonberg, Hensens, Hirshfield, Hoogsteen, Liesch, Springer, “Mevinolin: a highly potent competitive inhibitor of hydroxymethylglutaryl-coenzyme A reductase and a cholesterol-lowering agent,” Proc. Nat'l Acad. Sci. USA, 77: 3957-3961, 1980.

Arnold, Koning, Gallatti, Werchau, Konig, “Cytokine (IL-8, IL-6, TNF-alpha) and soluble TNF receptor-I release from human peripheral blood mononuclear cells after respiratory syncytial virus infection,” Immunol., 85:364-372, 1995.

Chiba, Takada, Miyamoto, Mitsui-Saito, Karaki, Misawa, “Augmented acetylcholine-induced, RhoA-mediated Ca²⁺sensitization of bronchial smooth muscle contraction in antigen induced airway hyperresponsiveness in rats,” Br. J. Pharmacol., 127:597-600, 1999.

Del Villar, Dorin, Sattler, Urano, Poullet, Robinson, Mitsuzawa, Tamanoi, “C-terminal motifs found in Ras-superfamily G-proteins: CAAX and C-seven motifs,” Biochem. Soc. Trans., 24:709-713, 1996.

Endo, Kuroda, Tsujita, ML-236A, ML-236B, and ML-236C, new inhibitors of cholesterogenesis produced by Penicillium citrinium,” J. Antibiot., 12:1346-1348, 1976.

Fisher, Johnson, Dillon, Parker, Graham, “Prophylaxis with respiratory syncytial virus F-specific humanized monoclonal antibody delays and moderately suppresses the native antibody response but does not impair immunity to late rechallenge.” J. Infect. Dis. 180:708-713, 1999.

Gong, Fujihara, Somlyo, Somlyo, “Translocation of rhoA associated with Ca²⁺ sensitization of smooth muscle,: J. Biol. Chem., 272:10704-10709, 1997.

Gong, Iizuka, Nixon, Browne, Hall, Eccleston, Sugai, Kobayashi, Somlyo, Somlyo, “Role of guanine nucleotide-binding-proteins-ras-family or trimeric proteins or both-in Ca²⁺sensitization of smooth muscle, Proc. Nat'l Acad. Sci., 93:1340-1345, 1996.

Graham, Perkins, Wright, Karzon, “Primary respiratory syncytial virus infection in mice,” J. Med. Virol., 26:153-162, 1988.

Graham, Davis, Tang, Bunton, Gruber, “Immunoprophylaxis and immunotherapy of respiratory syncytial virus-infected mice with RSV-specific immune serum.” Pediatr. Res. 34:167-172, 1993.

Graham, Tang, Gruber, “Topical immunoprophylaxis of respiratory syncytial virus (RSV)-challenged mice with RSV-specific immune globulin.” J. Infect. Dis. 171:1468-1474, 1995.

Gruber, Wilson, Throop, Wyde, “Immunoglobulin administration and ribavirin therapy: efficacy in respiratory syncytial virus infection of the cotton rat.” Pediatr. Res. 21:270-274, 1987.

Hall, “Rho GTPases and the actin cytoskeleton, Science, 279:509-514, 1998.

Hertz, Englund, Snover, Bitterman, McGlave, “Respiratory syncytial virus-induced acute lung injury in adult patients with bone marrow transplants: a clinical approach and review of the literature,” Med., 68:269-281, 1989.

Higgins and Casey, “The role of prenylation in G-protein assembly and function,” Cell. Sig., 8:433-437, 1996.

Hirata, Kikuchi, Sasaki, Kuroda, Kaibuchi, Matsuura, Seki, Saida, Takai, “Involvement of Rho p21 in the GTP-enhanced calcium ion sensitivity of smooth muscle contraction,” J. Biol. Chem., 267: 8719-22, 1992.

King, Jr., Burke, Clemens, et al., “Respiratory syncytial virus illness in human immunodeficiency virus and noninfected children,” Ped. Infect. Dis. J., 12:733-739, 1993.

Koch, Benz, Schmidt, Olenik, Aktories, “Role of Rho protein in lovastatin-induced breakdown of actin cytoskeleton,” J. Pharm. Exper. Ther., 283:901-909, 1997.

Kranenburg, Poland, Gebbink, Oomen, Moolenaar, “Dissociation of LPA-induced cytoskeletal contraction from stress fiber formation by differential localization of RhoA,” J. Cell Sci., 110(Pt 19):2417-2427, 1997.

MacDonald, Gerson, Kombrust, Kloss, Prahalada, Berry, Alberts, Bokelman, “Preclinical evaluation of lovastatin,” Am. J. Cardiol., 62: 16J-27J, 1988.

Maltese, Sheridan, Repko, Erdman, “Post-translational modification of low molecular mass GTP-binding proteins by isoprenoid,” Biol Chem., 265:2148-2155, 1990.

Narumiya, “The small GTPase Rho: cellular functions and signal transduction,” J. Biochem., 120:215-228, 1996.

Nguyen and Hildreth, “Evidence for budding of human immunodeficiency virus type 1 selectively from glycolipid-enriched membrane lipid rafts, J. Virol., 74:3264-3272, 2000.

Olmsted, Elango, Prince, Murphy, Johnson, Moss, Chanock, Collins, “Expression of the F glycoprotein of respiratory syncytial virus by a recombinant vaccinia virus:

comparison of the individual contributions of the F and G glycoproteins to host immunity,” Proc. Nat'l Acad. Sci., 83:7462-7466, 1986.

Overmeyer, Erdman, Maltese, “Membrane targeting via protein prenylation,” Meth. Mol. Biol., 88:249-263, 1998.

Park and Galper, “3-Hydroxy-3-methylglutaryl CoA reductase inhibitors up-regulate transforming growth factor-beta signaling in cultured heart cells via inhibition of geranylgeranylation of RhoA GTPase,” Proc. Nat'l Acad. Sci., 96:11525-30, 1999.

Parker, McNamara, Brown, “Plasma mevalonate as a measure of cholesterol synthesis in man,” J. Clin. Invest., 75:795, 1984.

Pastey, Crowe, Graham, “RhoA interacts with the fusion glycoprotein of respiratory syncytial virus and facilitates virus-induced syncytium formation,” J. Virol., 73:7262-7270, 1999.

Pastey, Gower, Spearman, Crowe, Graham, “A RhoA-derived peptide inhibits syncytium formation induced by respiratory syncytial virus and parainfluenza virus type 3,” Nat. Med., 6:35-40, 2000.

Ridley and Hall, “The small GTP binding protein rho regulates the assembly of focal adhesions and actin stress fibers in response to growth factors,” Cell, 70:389-399, 1992.

Saito, “Analysis of the cellular functions of the small GTP-binding protein rho p21 with Clostridium botulinum C3 exoenzyme,” Pharmacol. Japonica ,109:13-27, 1997.

Scheiffele, Rietveld, Wilk, Simons, “Influenza viruses select ordered lipid domains during budding from the plasma membrane,” J. Biol. Chem., 274:2038-2044, 1999.

Sekine, Fijuwara, Narumiya, “Asparagine residue in the rho gene product is the modification site for botulinum ADP-ribosyltransferase,” J. Biol. Chem., 264:8602-8605, 1989.

Sigurs, Bjarnason, Sigurbergsson, Kjellman, Bjorksten, “Asthma and immunoglobulin E antibodies after respiratory syncytial virus bronchiolitis: a prospective cohort study with matched controls. Pediatrics, 95:500-505, 1995.

Stein, Sherrill, Morgan, Holberg, Halonen, Taussig, Wright, Martinez, “Respiratory syncytial virus in early life and risk of wheeze and allergy by age 13 years,” Lancet, 354:541-545, 1999.

Takai et al., “Rho as a regulator of the cytoskeleton,” Trends Biochem, Sci., 20:227-231, 1995.

Treanor and Falsey, “Respiratory viral infections in the elderly,” Antiviral Res., 44:79-102, 1999.

Walsh and Graham, “Respiratory Syncytial Viruses in Viral infections of the respiratory tract for the series in lung biology,” In the Health and Disease, Dolin and Wright (eds), Marcel Dekker, Inc., NY, N.Y., 7(7):162-203, 1999.

Wendt, Fox, Hertz, “Paramyxovirus infection in lung transplant recipients,” J. Heart Lung Transplantation, 14:479-485, 1995.

Wendt and Hertz, “Respiratory syncytial virus and parainfluenza virus infections in the immunocompromised host,” Sem. Respiratory Infections, 10:224-231, 1995.

Wyde, Wilson, Petrella, Gilbert, “Efficacy of high dose-short duration ribavirin aerosol in the treatment of respiratory syncytial virus infected cotton rats and influenza B virus infected mice.” Antiviral Res. 1987; 7:211-220, 1987.

Zhang, Pekosz, Lamb, “Influenza virus assembly and lipid raft microdomains: a role for the cytoplasmic tails of the spike glycoproteins,” J. Virol., 74:4634-4644, 2000.

What is claimed is:

1. A method of inhibiting infection of a cell by a virus in a subject comprising the step of administering to said subject an inhibitor of HMG-CoA reductase.

2. The method of claim 1, wherein said subject does not have an existing viral infection.

3. The method of claim 2, wherein said subject is or will become immunosuppressed.

4. The method of claim 3, wherein said subject suffers from severe combined immundeficiency.

5. The method of claim 3, wherein said subject is taking or will take immunosuppressive drugs.

6. The method of claim 5, wherein said subject is or will be a transplant recipient.

7. The method of claim 1, wherein said subject has an existing viral infection.

8. The method of claim 1, wherein said virus is selected from the group consisting of human immunodeficiency virus, human respiratory syncytial virus (RSV), parainfluenza virus, ebola virus, measles, canine distemper, Newcastle's Disease virus, bovine RSV, ovine RSV and turkey rhinotracheitis.

9. The method of claim 1, wherein said subject is a human.

10. The method of claim 9, wherein said virus is human immunodeficiency virus type 1.

11. The method of claim 9, wherein said virus is respiratory syncytial virus.

12. The method of claim 9, wherein said virus is human parainfluenza virus type 3.

13. The method of claim 1, wherein said inhibitor is selected from the group consisting of lovastatin, simvastatin, fluvastatin, atorvastatin, pravastatin and mevastatin.

14. The method of claim 1, further comprising administering to said subject an inhibitor of isoprenylation that is distinct from said HMG-CoA reductase inhibitor.

15. The method of claim 10, further comprising administering to said subject a nucleoside analog composition.

16. The method of claim 15, further comprising administering to said subject a protease inhibitor.

17. The method of claim 11, further comprising administering to said subject an antibody composition that binding immunologically to RSV.

18. The method of claim 11, further comprising administering to said subject ribavarin.

19. The method of claim 1, wherein said subject is a non-human mammal.

20. The method of claim 19, wherein said non-human mammal is a livestock animal.

21. A method of inhibiting infection of a cell by a virus in a subject comprising the step of administering to said subject an inhibitor of isoprenylation.

22. The method of claim 21, wherein said subject does not have an existing viral infection.

23. The method of claim 22, wherein said subject is or will become immunosuppressed.

24. The method of claim 23, wherein said subject suffers from severe, combined immundeficiency.

25. The method of claim 23, wherein said subject is taking or will take immunosuppressive drugs.

26. The method of claim 25, wherein said subject is or will be a transplant recipient.

27. The method of claim 21, wherein said subject has an existing viral infection.

28. The method of claim 21, wherein said virus is selected from the group consisting of human immunodeficiency virus, human respiratory syncytial virus (RSV), parainfluenza virus, ebola virus, measles, canine distemper, Newcastle's Disease virus, bovine RSV, ovine RSV and turkey rhinotracheitis.

29. The method of claim 21, wherein said subject is a human.

30. The method of claim 29, wherein said virus is human immunodeficiency virus type 1.

31. The method of claim 29, wherein said virus is respiratory syncytial virus.

32. The method of claim 29, wherein said virus is human parainfluenza virus type 3.

33. The method of claim 21, wherein said inhibitor is selected from the group consisting of L-739,749, L-744,832, SCH 44342 (1-(4-pyridylacetyl)-4-(8-chloro-5,6 dihydro-IIH benzo [5,6] cyclohepta [1,2-b]pyridin-11-yhdene) piperidine), BZA-5B, FTI-276 and B1086.

34. The method of claim 21, further comprising administering to said subject an inhibitor of HMG-CoA reductase that is distinct from said isoprenylation inhibitor.

35. The method of claim 30, further comprising administering to said subject a AZT composition.

36. The method of claim 35, further comprising administering to said subject a protease inhibitor.

37. The method of claim 31, further comprising further comprising administering to said subject an antibody composition that binding immunologically to RSV.

38. The method of claim 32, further comprising further comprising administering to said subject ribavarin.

39. The method of claim 21, wherein said subject is a non-human mammal.

40. The method of claim 39, wherein said non-human mammal is a livestock animal.

41. A composition comprising:
(a) an HMG-CoA reductase inhibitor; and
(b) an antiviral drug.

42. The composition of claim 41, wherein said inhibitor is lovastatin, simvastatin, fluvastatin, atorvastatin, pravastatin and mevastatin.

42. The composition of claim 41, wherein said inhibitor is lovastatin, simvastatin, fluvastatin, atorvastatin, pravastatin and mevastatin.

43. The composition of claim 41, wherein said antiviral drug is a nucleoside analog, a protease inhibitor, a non-nucleoside reverse transcriptase inhibitor, and integrase inhibitor or a viral entry inhibitor.

44. A composition comprising:
(a) an isoprenylation inhibitor; and
(b) an antiviral drug.

45. The composition of claim 44, wherein said inhibitor is selected from the group consisting of L-739,749, L-744,832, SCH 44342 (1-(4-pyridylacetyl)-4-(8-chloro-5,6 dihydro-IIH benzo [5,6] cyclohepta [1,2-b]pyridin-11-yhdene) piperidine), BZA-5B, FTI-276 and B1086.

46. The composition of claim 44, wherein said antiviral drug is a nucleoside analog, a protease inhibitor, a non-nucleoside reverse transcriptase inhibitor, and integrase inhibitor or a viral entry inhibitor.

47. A method of preventing the emergence of viral resistance in a subject comprising administering to said subject a combination drug therapy, wherein one of said drugs is an inhibitor of HMG-CoA reductase.

48. A method of preventing the emergence of viral resistance in a subject comprising administering to said subject a combination drug therapy, wherein one of said drugs is an inhibitor of isoprenylation.

49. A method of treating a drug-resistant viral infection in a subject comprising administering to said subject a combination drug therapy, wherein one of said drugs is an inhibitor of HMG-CoA reductase.

50. A method of treating a drug-resistant viral infection in a subject comprising administering to said subject a combination drug therapy, wherein one of said drugs is an inhibitor of isoprenylation.
